(19) United States
(12) Patent Application Publication
(10) Pub. No.: US 2004/0258577 A1
(43) Pub. Date: Dec. 23, 2004

(54) Disinfection device for an air ventilator

(76) Inventor: Ting-Hsing Chen, Tainan Hsien (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/462,633

(22) Filed: Jun. 17, 2003

(51) Int. Cl. A61L 9/20

(52) U.S. Cl. 422/121

(57) ABSTRACT

A disinfection device for an air ventilator includes a chassis, a lid and a light media layer. The chassis has a recess at the rear end to accommodate the lid, and both the chassis and the lid comprise air inhales. The recess of the chassis comprises an ultraviolet ray tube therein. Inner surfaces of the recess of the chassis and inner surfaces of the lid are applied with the light media layer thereon. A filter is provided between the air inhale of the chassis and the air inhale of the lid to achieve double effects of disinfection and eliminating germs.

### FIELD OF THE INVENTION

This invention relates to an electronic disinfection device, and more particularly to a device with ultraviolet ray to eliminate germs spread in the air.

### BACKGROUND OF THE INVENTION

Electronic fans are widely used by consumers in summer season at home or any sealed room, however a sealed room may easily get germs and produce mold, thus the room is always filled with odor smell, and the fans make the situation even worse.

In order to solve this problem, inventors have provided with a light media, which includes TiO₂ to eliminate germs and mold (it is known that ultraviolet ray is the best media for this purpose), however, ultraviolet ray is harmful to human beings to dispose under the ultraviolet ray for a certain time.

In view of this and many other shortcomings, a renovated device is derived, which keeps the ultraviolet ray in an enclosed area to prevent from direct ejecting onto human bodies while it maintains the function of eliminating any germs and mold from accumulating within the room.

### SUMMARY OF THE INVENTION

It is the primary object of the present invention to provide a disinfection device for an air ventilator, which can eliminate germs and mold effectively.

It is another object of the present invention to provide the disinfection device for an air ventilator, which protects consumers from being shined by the ultraviolet ray directly.

It is a further object of the present invention to provide the disinfection device for an air ventilator, which is inexpensive in manufacture and easy to operate.

### BRIEF DESCRIPTION OF THE DRAWINGS

FIG. 1 is a perspective view of a first embodiment of the present invention;

FIG. 2 is a side cross-sectional view of the first embodiment;

FIG. 3 is an exploded view of a second embodiment of the present invention, and

FIG. 4 is a side cross-sectional view of the second embodiment.

### DETAILED DESCRIPTION OF THE PREFERRED EMBODIMENTS

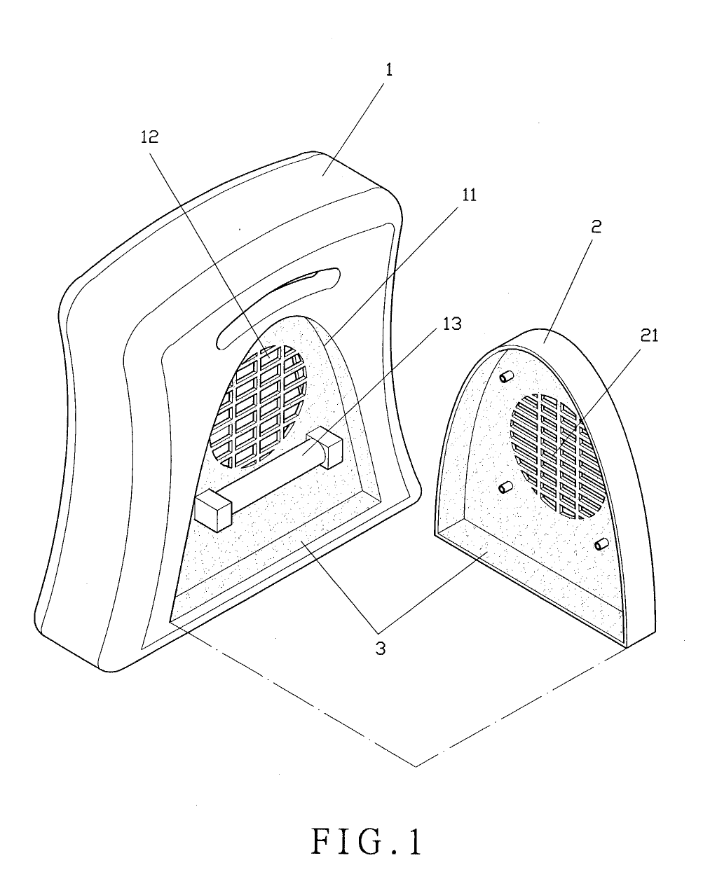

A disinfection device for an air ventilator of the present invention, as shown in FIG. 1, comprises a chassis 1, a lid 2 and a light media layer 3.

The chassis 1 has a recess 11 at the rear end to receive the lid 2 therein. Both the chassis 1 and the lid 2 comprise air inhales 12 and 21. The recess 11 accommodates an ultraviolet ray tube 13 therein (it is to be noted that near ultraviolet ray can provide the best effect). The inner surfaces of the recess 11 of the chassis 1 and the inner surfaces of the lid 2 comprise the light media layer 3, which are to spread or to brush TiO₂ onto the surfaces.

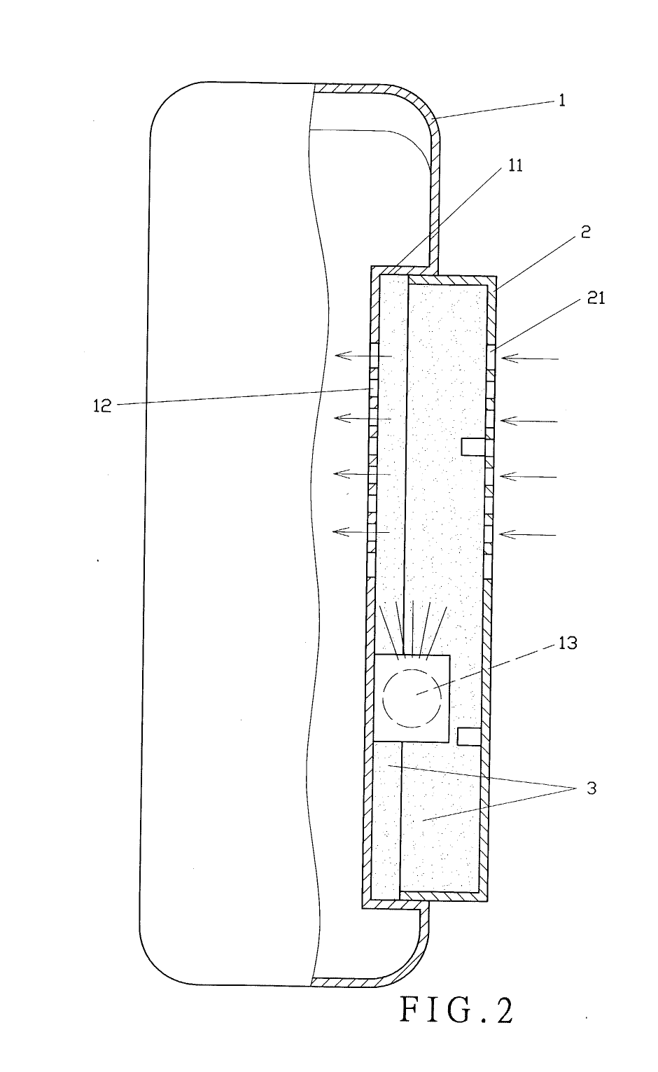

When operating the air ventilator of the present invention, as shown in FIG. 2, air flows through the inhales 12 and 21 and the recess 11 whereas the light media layer 3 on the inner surfaces of the recess 11 and the lid 2 will be tempted by the ultraviolet ray tube 13 to eliminate mold, germs and odor smell. The ultraviolet ray tube 13 is secured behind the chassis 1 and covered by the lid 2, which blocks the ray from shining to a user directly and minimizes harm to a human body.

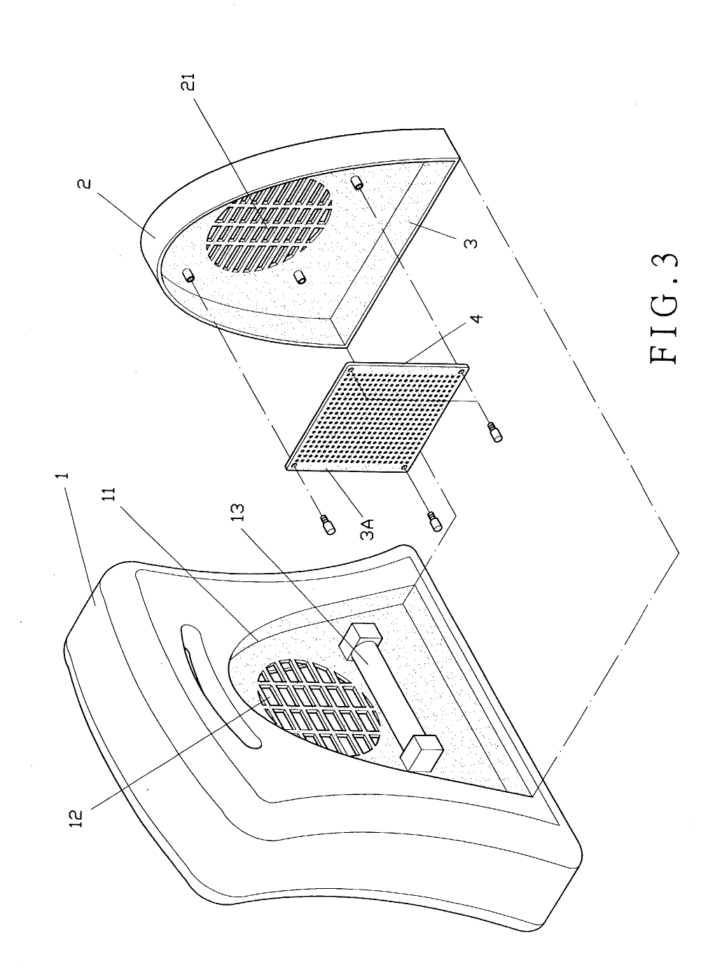

FIG. 3 shows a second embodiment of the present invention, which comprises a chassis 1, a lid 2, a light media layer 3 and a filter 4.

The chassis 1 has a recess 11 at the rear end to receive the lid 2 therein. Both the chassis 1 and the lid 2 comprise air inhales 12 and 21. The recess 11 accommodates an ultraviolet ray tube 13 therein. Between the air inhales 12 and 21 of the chassis 1 and the lid 2 is a filter 4 applied with a light media layer 3A. Both of the filter 4 and the light media layer 3A are made of metallic grid or non-fabric material soaking in TiO₂.

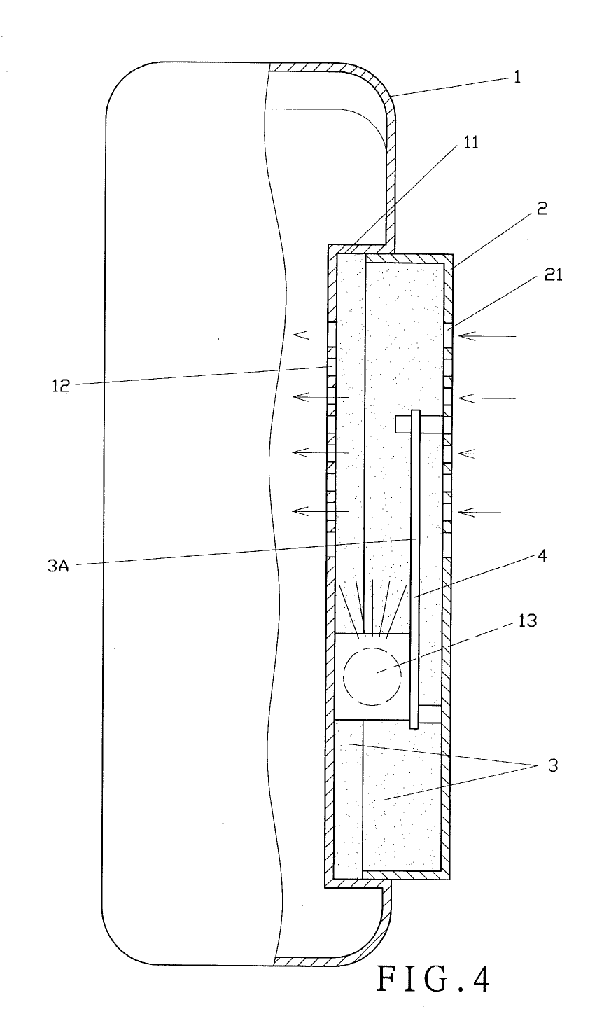

When air flows through the air inhales 12 and 21, the recess 11 and the filter 4 of the present invention, as shown in FIG. 4, the light media layer 3A is tempted by the ultraviolet ray tube 13 to eliminate mold, germs and odor smell.

I claim:

1. A disinfection device for an air ventilator comprising a chassis, a lid and a light media layer, wherein said chassis comprising a recess at a rear end adapted for said lid to be secured therein, both said chassis and said lid comprising air inhales therein, said recess of said chassis having an ultraviolet ray tube therein, inner surfaces of said recess of said chassis and inner surfaces of said lid being applied with said light media layer thereon.

2. The disinfection device for an air ventilator, as recited in claim 1, wherein between said inhale of said chassis and said inhale of said lid is a filter applied with a light media layer thereon.
